(19) United States
(12) Patent Application Publication
(10) Pub. No.: US 2006/0178615 A1
(43) Pub. Date: Aug. 10, 2006

(54) Allergy tester

(76) Inventors: Steen Meier Ronborg, Bagsvaerd (DK); Erik Meier Mortensen, Praesto (DK)

Correspondence Address: GIFFORD, KRASS, GROH, SPRINKLE & CITKOWSKI, P.C, PO BOX 7021, TROY, MI, 48007-7021, US

(21) Appl. No.: 10/558,943

(22) PCT Filed: May 28, 2004
(86) PCT No.: PCT/DK04/00375

§ 371 Date: Feb. 2, 2006

Related U.S. Application Data
(60) Provisional application No. 60/480,734, filed on Jun. 24, 2003.

(30) Foreign Application Priority Data
May 30, 2003 (DK) PA 2003 00813

(51) Int. Cl. A61B 17/20; A61M 37/00

(52) U.S. Cl. 604/46

(57) ABSTRACT

The present invention relates to a device for delivering a medicament or a diagnostic agent to the skin or mucosa of an animal such as a human being, wherein a chamber filled with the medicament or the diagnostic agent is separate from the housing with a rod capable of transferring the medicament or the diagnostic agent to the animal. The chamber is connected to the housing with the rod before transfer of medicament or diagnostic agent. In particular the invention relates to a device for delivering allergens in allergy tests.

The present invention relates to a device for delivering a medicament or a diagnostic agent to the skin or mucosa of an animal such as a human being, wherein a chamber filled with the medicament or the diagnostic agent is separate from the housing with a rod capable of transferring the medicament or the diagnostic agent to the animal. In particular the invention relates to a device for delivering allergens in allergy tests.

### BACKGROUND

In recent years, allergies have become an increasing problem especially in the Western World. Therefore, the need for being able to diagnose such diseases quickly, securely and reproducibly is growing steadily.

Conventionally, allergies are diagnosed by manually applying a number of different allergen solutions on the palm side of the patient's forearm. After this, an injection needle is pricked through the individual drops and about 1 mm into the skin so that a cavity is formed for absorbing some of the solution.

If the person is allergic to an allergen solution, the skin will after 10 to 15 minutes become red and swollen around the injection spot to an extent that depends on the quantity or the concentration of the solution that can be absorbed by the cavity. The degree of the allergic reaction can be given as a function of the extent of the swelling.

It is very difficult to dose the solution quantity with great accuracy, however, as the insertion depth of the needle and thereby the cubic capacity of the, cavity depends solely on the operator's routine and skill.

With the above conventional method, the degree of the allergic reaction can therefore not be estimated with the wanted accuracy and reproducibility.

As various allergen extracts are often used at tests of an allergic reaction, there is moreover an added risk of confusing the test solutions. Furthermore, the solutions are placed directly on the skin, which opens up the possibility of contamination with allergizing agents that might give a false positive response. By using this open handling of the allergen extracts, there is furthermore a risk of the operator becoming sensitized to one of the respective allergen extracts.

An additional problem of the conventional method is the risk of infection which is present to the operator in that the injection needle remains unprotected after use. U.S. Pat. No. 5,099,857 discloses a construction with a mowable injection needle and a sealed capsule containing an allergen solution. At injection, the needle will rupture the capsule, and the solution will therefore run out and form a drop on the skin, after which the needle via the drop will penetrate the skin in the same way as mentioned above. Also in this case, the estimates of the possible skin reaction will be inaccurate. Thus, there is a need for improvements in such devices.

### SUMMARY

The present invention provides a solution to prior art problems by providing a device for delivering a medicament or a diagnostic agent to the skin or mucosa of an animal, said device comprising
at least one rod house and at least one separate chamber house, wherein said chamber house is capable of being connected to the rod house,
wherein the rod house comprises at least one housing, said at least one housing having a distal end and a proximal end, and at least one rod, said rod having a distal end and at least one proximal end, and said rod being slidably arranged in the housing, said rod being capable of being activated by being pushed towards the proximal end of the housing, and
wherein the chamber house comprises at least one chamber, a first wall of said chamber being a first sealing and a second wall of said chamber being a second sealing, said first sealing and said second sealing being arranged so that an axis through said chamber may intersect both sealings, and said chamber comprising said medicament or said diagnostic agent.

By the device according to the invention, the medicament or diagnostic agent is enclosed in the chamber until activation of the rod, whereby the risk of contamination of the environment is eliminated. Furthermore, in particular when testing for allergy, it is of importance that the various allergens are not mixed, a problem that is reduced with the present invention. Also, the device presents a solution to storage problems, since the chamber house may be stored separate from the rod house, and the chamber is the only part needing refrigerated storage.

The device according to the invention provides delivery of the medicament or diagnostic agent easily, quickly, with less pain and greater accuracy and reproducibility than prior art devices.

In another aspect the invention relates to a method for delivering a sufficient amount of medicament or diagnostic agent to an animal in need thereof, comprising
arranging a device as defined above, wherein the chamber house comprising the medicament or diagnostic agent is connected to the rod house, adjacent the skin or mucosa of said animal,
activating the rod of the device, thereby delivering said medicament or diagnostic agent to said animal.

In particular the method is suitable for allergy tests.

Furthermore, the invention relates to a separate chamber house for being connected to a rod house as defined above, wherein the chamber house comprises a chamber, a first wall of said chamber being a first sealing and a second wall of said chamber being a second sealing, said first sealing and said second sealing being arranged so that an axis through said chamber may intersect both sealings, and said chamber comprising a medicament or a diagnostic agent. In particular, the chamber house may be connected to the rod house so that the rod may penetrate both sealings through said axis.

Also, the invention relates to a separate rod house comprising at least one housing, said at least one housing having a distal end and a proximal end, and at least one rod, said rod having a distal end and at least one proximal end, and said rod being slidably arranged in the housing, said rod being capable of being activated by being pushed towards the proximal end of the housing, and said rod house being capable of being connected to a chamber house.

In yet another aspect the invention relates to a device for delivering a medicament or a diagnostic agent to the skin or mucosa of an animal, said device comprising
at least one rod house and at least one chamber house, wherein the rod house comprises at least one housing, said at least one housing having a distal end and a proximal end, and at least one rod, said rod having a distal end and at least one proximal end, and said rod being slidably arranged in the housing, said rod being capable of being activated by being pushed towards the proximal end of the housing,
wherein the chamber house comprises at least one chamber, a first wall of said chamber being a first sealing and a second wall of said chamber being a second sealing, said first sealing and said second sealing being arranged so that an axis through said chamber may intersect both sealings, and said chamber comprising said medicament or said diagnostic agent, and
wherein the chamber house is provided with a labelling means for transferring a label to the animal being tested.

### DRAWINGS

FIG. 1 shows an assembled device according to the invention.

FIG. 2 shows a rod.

FIG. 3 shows the cap of the rod house.

FIG. 4 shows a locker bushing of the rod house.

FIG. 5 shows a proximal bushing of the rod house.

FIG. 6 shows a marking means.

FIG. 7 shows a chamber house.

FIG. 8 shows a plug.

FIG. 9 shows a pressing aid.

FIGS. 10a, 10b, and 10c illustrate the different positions of the rod before, during and after activation.

FIGS. 11a, 11b and 11c show one embodiment of a multidevice composed of ten single devices.

FIG. 12 shows another embodiment of a multidevice having three separate rod houses and one chamber house with three chambers.

FIG. 13 shows an assembled device according to the invention.

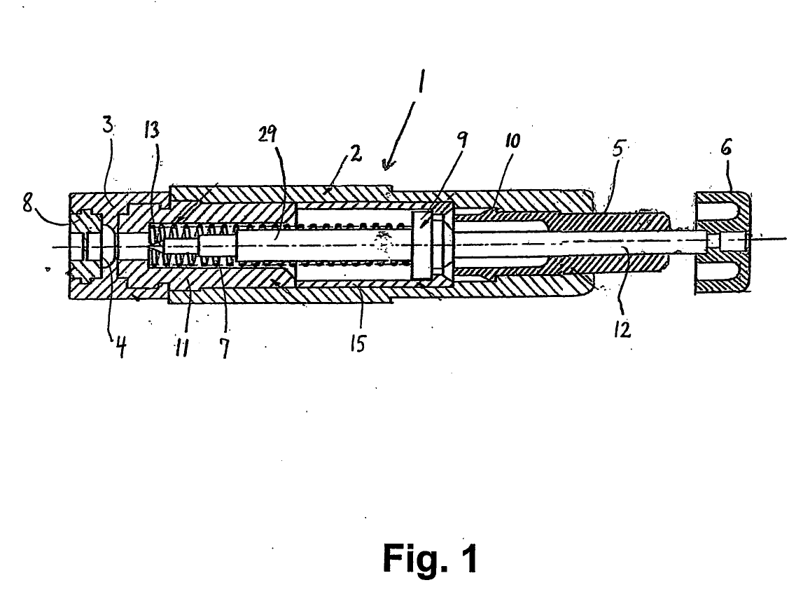

### DETAILED DESCRIPTION OF THE INVENTION

The present invention relates to a device for delivering a medicament or a diagnostic agent to the skin or mucosa of an animal. In particular the device is suitable for allergy tests, wherein an amount of an allergen is delivered into the skin or mucosa of the animal to be tested and the animal's reaction to the allergen is monitored.

The animal may be any animal, in particular a mammal, such as a horse, a dog, and a cat; more specifically the mammal is a human being.

The device according to the invention comprises two separate components, namely at least one rod house and at least one separate chamber house, wherein said at least one chamber house is capable of being connected to the at least one rod house.

By constructing the device with two separate components the device fulfils several needs. First, during storage only the chamber house need cooled storage, since it is only the chamber house that comprises the allergen. Thereby the need for specialised storage has been reduced compared to prior art devices. Furthermore, the two components, rod house and chamber house, maybe produced at separate locations, and need not be connected until immediately before use. This offers a great freedom in production logistics, since the allergen may be produced locally, and filled into the chamber house and subsequently assembled with the rod house.

Rod House

The rod house comprises at least one housing, wherein said at least one housing has a distal end and a proximal end, and at least one rod, and said rod has a distal end and at least one proximal end. Said rod is slidably arranged in the housing, whereby the term “slidably” means that the rod may be slid towards the proximal end of the housing. During use the rod is capable of being activated by being pushed towards the proximal end of the housing. In a preferred embodiment the rod is a longitudinal rod arranged axially in the rod house. By the longitudinal axial arrangement precision of the transfer of the medicament or diagnostic agent is increased, in that any play of the rod in the housing is reduced to a minimum.

The proximal end of the rod house is preferably sealed by a rod house sealing. The sealing may be a stationary sealing to be penetrated by the needle during use, or a removable sealing, whereby the sealing is removed when the rod house is assembled with the chamber house. It is preferred that the sealing is stationary, for example as being moulded together with the proximal part of the rod housing.

In a preferred embodiment the distal end of the rod is projecting out of the distal end of the rod house, whereby the rod may be activated by pressing the distal end of the rod towards the proximal end of the rod house. Preferably the rod house comprises means for retracting the rod after activation to ensure that the needle is retracted into the chamber house or rod house. The means for retracting the rod may be any suitable means, such as a spring or a pump. Preferably the means for retracting the rod is a spring.

To be able to predetermine how deep into the skin or mucosa of the individual the needle may travel, the housing preferably comprises stopper means for stopping advance of the rod at a predetermined position during activation, the stopper means may be any suitable means, such as a mechanical stop per means, such as wherein the stopper means is a shoulder in the housing dimensioned to engage a shoulder on the rod.

The rod house is made from any suitable material, preferably a plastic material capable of being moulded, such as injection moulded. The rod house may be formed in one part or constructed from two or more parts assembled to form the rod house.

An example of materials selected for the rod house is polypropylene, such as a polypropylene having the following properties:

| | Typical value | Unit | Test method |
|---|---|---|---|
| Tensile modulus | 1300-1400 | MPa | ISO 527-2 |
| Tensile stress at yield | 20-30 | MPa | ISO 527-2 |
| Tensile strain at yield | 4-8 | % | ISO 527-2 |
| Rockwell hardness | 80-90 | R-scale | ISO 2039-2 |

In particular, the polypropylene is BC245MO from Borealis ANS, Denmark.

Needle

The proximal end of the rod is provided with means for transferring the medicament or the diagnostic agent to the animal. This is preferably arranged by providing the proximal end of the rod with a tapering end, either by integrating a needle in the proximal end or by forming the proximal end of the rod as a needle.

In order to ensure transfer of a sufficient amount of medicament or diagnostic agent, the proximal end of the rod is preferably provided with at least one recess.

The at least one recess may be arranged in any suitable part of the proximal end of the rod, such as in the tapering portion of the needle, the only requirement being the medicament or diagnostic agent in the recess is transferred to the animal during normal transfer. The recess may have its opening into the tip of the rod or into the longitudinal part of the rod.

In a preferred embodiment the proximal end of the rod is provided with more than one recess, such as at least two recesses, for example at least four recesses.

The rod material is preferably a material having a high strength. In particular when the needle is made from the same material as the rest of the rod.

Examples of materials for the rod are polypropylene, polypropylene mixed with glass fibre, polyamide metals and metal alloys, and mixtures thereof.

Since the needle penetrates at least two sealings before penetrating the skin, it is necessary that the needle maintains the shape and is not blunted during penetration of the sealings. Accordingly, in a preferred embodiment, the requirements to the needle material are at least the following mechanical properties:

| | Typical value | Unit | Standard |
|---|---|---|---|
| Compressive strength | 110-130 | MPa | ASTM D695 |
| Shear strength | 50-70 | MPa | ASTM D732 |
| Taber abrasion, CS-17, | 20-25 | mg/1000cy | GE |
| 1 kg | | | |
| Tensile stress at break, | 110-140 | MPa | ISO 527 |
| 5 mm/min | | | |
| Tensile strain at break, | 1.7-2.5 | % | ISO 527 |
| 5 mm/min | | | |
| Tensile modulus, 1 mm/min | 9000-11000 | MPa | ISO 527 |
| Flexural strength at break, | 200-250 | MPa | ISO 178 |
| 2 mm/min | | | |
| Flexural modulus, | 9000-10000 | MPa | ISO 178 |
| 2 mm/min | | | |
| Hardness, H358/30 | 100-130 | MPa | ISO 2039/1 |
| Hardness, Rockwell R | 100-130 | — | ISO 2039/2 |

In a preferred embodiment, the needle is made from Valox® from General Electric Plastics B.V., or from Miramido H3KC, Plastcom ANS, Denmark.

In one aspect the invention relates to a rod house separate from a chamber house. The rod house is as described above. The separate rod house may be produced and sold separate from the chamber house for being connected before use.

Chamber House

The chamber house comprises a chamber, wherein a first wall of said chamber is a first sealing and a second wall of said chamber is a second sealing, said first sealing and said second sealing are arranged so that an axis through said chamber may intersect both sealings, and said chamber comprising said medicament or said diagnostic agent.

As described above it is an advantage that the chamber house is separate from the rod house. During use the chamber house is either arranged adjacent the rod house or attached to the rod house. The chamber house is arranged in relation to the rod house so that the rod penetrates both sealings during use along said axis. Accordingly, the chamber house may be connected to the rod house so that the proximal end of the rod penetrates the first sealing and the second sealing when slid proximally.

In a preferred embodiment the chamber house has means for being attached to the rod house. The chamber house may be attached to the rod house either releasably or non-releasably by any suitable means. In one embodiment the distal part of the chamber house is provided with adhesive material, so that the chamber house and rod house are connected by means of the adhesive material. In another embodiment the chamber house and the rod house are connected through mechanical means, such as mechanical means selected from a thread, a luer lock, a bajonet lock, and a snap fit lock. In a preferred embodiment the chamber house are attached to the rod house through a snap fit lock.

The first sealing and the second sealing are attached to the chamber house by any suitable means. In one embodiment at least the first sealing is integrated with the walls of the chamber house, for example being moulded together with the walls of the chamber house.

In a preferred embodiment the second sealing is releasably attached to the chamber house, such as by means of a plug inserted into the chamber house or by means of a cover. By closing the chamber of the chamber house with a plug or a cover, the filling of the chamber is facilitated. After sealing the chamber with the plug or the cover, the medicament of diagnostic agent is enclosed in the chamber reducing the risk of atomizing of the medicament or diagnostic agent thereby reducing the risk of contamination of the environment.

The chamber house may be formed from any suitable material, capable of being produced in a cost effective manner. Furthermore, the chamber house material must not react with the medicament or diagnostic agent.

Preferably the chamber house is made from a plastic material, more preferably from a resilient material. In particular when the chamber house is attached to the rod house be means of a snap fit lock it is desired that the material is resilient. Furthermore, the resilient material offers a more pleasant feeling to the individual being tested when the chamber house is arranged on the skin or mucosa. Also the sealings are preferably produced from a resilient plastic material. If the chamber house is produced from a harder material than the sealing, the chamber house may be produced for example by two-component moulding.

Examples of the chamber house material are a thermoplastic material, such as a rubber, a polyolefin or mixtures thereof, in particular a plastic material having the following characteristics:

| | Typical value | Unit | Standard |
|---|---|---|---|
| Hardness | 35-50 | Shore A | ASTM D2240 |
| Density | 0.9-1.1 | g/cm³ | ASTM D792 |
| Tensile strength | 3.0-5.0 | MPa | ASTM D638 |
| E-100 | 0.5-0.7 | MPa | ASTM D638 |
| E-300 | 1.3-1.8 | MPa | ASTM D638 |
| Elongation at break | 500-600 | % | ASTM D638 |
| Tear strength | 12-20 | kN/m | ASTM D624 |

In particular, the chamber house material may be Dryflex® from Nolato Elastoteknik AB, Sweden.

The plug may be made from the same material as the chamber house or from another material, preferably a thermoplastic material. In particular the plug may be made from a natural or synthetic rubber, such as a material having the following characteristics:

| | Typical value | Unit | Standard |
|---|---|---|---|
| Hardness | 94 | Shore A | DIN 53505 |
| Density | 1.13 | g/cm³ | DIN 53479 |
| Tensile strength | 14.5 | N/mm² | DIN 53504 |
| Elongation at break | 610 | % | DIN 53504 |
| Modulus at 100% | 6.0 | N/mm² | DIN 53504 |
| Modulus at 200% | 6.6 | N/mm² | DIN 53504 |
| Modulus at 300% | 7.5 | N/mm² | DIN 53504 |

Most preferably, the plug is made from Thermoplast K TF9AAE from Kraiburg TPE GmbH, Germany.

The cover may also be made from any suitable material, for example made from a foil, such as a metal foil, and being welded to the chamber house.

In one aspect, the invention relates to a chamber house for being connected to a rod house as defined above, wherein the chamber house is as defined above. The chamber house may be sold separate from the rod house for being connected immediately before use. The at least one chamber may be prefilled at least partly with medicament or diagnostic agent, such as prefilled with an allergen or a combination of allergens.

In another embodiment the camber house is sold empty to be filled with medicament or diagnostic agent locally before use. This is especially relevant when using with allergens of only local relevance.

Marking Means

To increase the security of the device, it is preferably provided with marking means to mark that the rod has been activated, whereby accidental re-use of the device is avoided. The marking means may be any suitable means such as change of physical appearance in at least a part of the device, such as change of colour and/or change of shape.

In a preferred embodiment the marking means is a marker projecting from the distal end of the housing and said marking means is activated when the rod is activated. For example the at least one marking means is a marker being arranged concentrically around at least a part of the distal end of the rod. Thereby the marking means is pressed forwards towards the proximal end of the device when the rod is activated. Furthermore, it is preferred that once the marker means has been activated it takes a new position or changes colour. For example in one embodiment the housing comprises means for engaging the marker means when the marker means is activated, thereby holding the marker means in a new position after activation.

The marking means may be made from any suitable material; however it is preferred that the marker means has colour different from the colour of the distal end of the rod.

Multidevice

The device according to the invention may be a device constructed to transfer one type of medicament or diagnostic agent. However, in another embodiment the device according to the invention may be a multidevice or part of a multidevice, wherein the term multidevice means a device comprising two or more different medicaments and/or diagnostic agents.

In particular when the device is used for transfer of diagnostic agents, such as allergens in an allergy testing, a series of different allergens is normally used. Thus, it is advantageous to arrange the device as a multidevice having the number of chambers with medicament or diagnostic agent corresponding to the number of different medicaments or diagnostic agents in the series, whereby all medicaments or diagnostic agents may be transferred at once. Furthermore, a multidevice may secure the order in which the medicament or diagnostic agent is transferred.

A multidevice may be constructed by arranging two or more chamber houses as described above, as well as either two or more rods, or a rod with two or more needles capable of transferring the medicament and/or diagnostic agent from the two or more chambers. In order to assure the security of the device, it must be assured that a needle only transfers one medicament or diagnostic agent, to avoid mixing of medicaments or diagnostic agents.

In one embodiment the device comprises at least two chamber houses, such as at least three chamber houses. The chamber may be separate from each other, however in a preferred embodiment the at least two chamber houses are connected.

In one embodiment, with respect to a multidevice, the rod has at least two proximal ends, such as at least three proximal ends. Such a rod having more than one proximal end, may be have one common distal end.

In another embodiment the rod house comprises the same number of rods as the number of chambers. Thus, in one embodiment the rod house comprises at least two rods, such as at least three rods.

When a multidevice comprises two or more rods, it may be advantageous to apply a common activation means to the rods, for example to be able to activate all rods in one operation. Accordingly, in one embodiment two or more rods are connected at their distal ends to a common activation means, such as a connector cap.

Labelling Means

The device according to the invention is in one embodiment provided with a labelling means, so that the skin or the mucosa of said mammal is labelled when the medicament or diagnostic agent is delivered to the mammal. The labelling means may be applied to the skin or mucosa before, during or after transfer of the medicament or diagnostic agent.

The labelling means may be any suitable labelling, such as a tape or a colour or letters or figures, or a combination thereof, such as a code identifying the content of the chamber and/or the individual being tested. The labelling means is preferably arranged on the chamber house, so that it is transferred during use of the device, for example by a stamping effect when the labelling means is arranged on the proximal part of the chamber house.

Use

The device to the invention is suitable for delivering a medicament or a diagnostic agent to the skin or mucosa of an animal, such as human being.

Accordingly, one aspect of the invention relates to a method for delivering a sufficient amount of medicament or diagnostic agent to a mammal in need thereof, comprising
arranging a device as defined above, wherein the chamber house comprising the medicament or diagnostic agent is connected to the rod house, adjacent the skin or mucosa of said animal,
activating the rod of the device, thereby delivering said medicament or diagnostic agent to said animal.

The device and method is suitable for delivering any kind of medicament or diagnostic agent to the skin or mucosa of the animal.

In relation to medicaments, the invention in particular relates to delivery of vaccines, probiotics, antibiotics or vitamins, preferably to delivery of vaccines.

In relation to diagnostic agents, the invention in particular relates to delivery of allergens for allergy tests. In this relation the chamber house comprises an allergen or combination of allergens.

In one embodiment the devices are used singly for delivering the determined medicaments or diagnostic agents, it is however preferred when delivering allergens that the device is arranged as a multidevice for delivering the series of allergens necessary for the testing in one application round.

The allergens used may be any allergens used for testing for allergy, such as pollen from various plants, moulds, insect venoms, food allergens, house dust mite allergens, and animal hair. In relation to individuals to be treated in a hospital it may be necessary to test for latex allergy, in particular when treating unconscious individuals before for example an operation. For latex allergy test a multidevice comprising three chambers is suitably used, wherein one chamber comprises latex allergen, one chamber comprises saline, and one chamber comprises histamine, the latter two being negative and positive controls, respectively. For latex allergy tests the three chambers are preferably arranged in chamber houses being connected.

### REFERENCE SIGNS IN DRAWINGS

1 Device according to the invention
2 Housing
3 Chamber house
4 Chamber
5 Marker means
6 Cap
7 Spring
8 Plug
9 Projecting means
10 Projecting means on marker means
11 Bushing
12 Distal end of rod
13 Needle
14 Shoulder on rod
15 Internal bushing
16 Opening
17 Recess in chamber house
18 First sealing
19 Recess for receiving a plug
20 Recess for receiving distal end of the rod
21 Connector
22 Bracelet
23 Opening for receiving a device according to the invention
24 Recess for receiving bracelet
25 Connector cap
26 Package with three devices according to the invention
27 Holder
28 Connector cap
29 Rod
30 Recess in tip of rod
31 Rib on housing
32 Projecting part on bushing
33 Second sealing
34 Stem
35 Rod house sealing
36 Cover

### DETAILED DESCRIPTION OF THE INVENTION IN RELATION TO THE DRAWINGS

In the following, one embodiment of the invention is described in relation to the drawings. In FIG. I a device 1 according to the invention is shown. The device 1 is assembled of a rod house comprising a housing 2 and a rod 29, and a chamber house 3 comprising chamber 4. In the shown embodiment the rod house consists of housing 2, an internal bushing 15, and a bushing 11. The rod house further encloses rod 29 arranged axially in the housing 2, spring 7 arranged concentrically around rod 29, as well as marker means 5 arranged concentrically around the distal end 12 of the rod 29. Furthermore, the distal end of the rod 29 is provided with a cap 6 facilitating the activation of the rod 29. In the proximal end of the device 1 the chamber house 3 is connected to the rod house through a snap fit lock to the bushing 11. The chamber house 3 is provided with a plug 8, defining a chamber 4 between one wall of the chamber house 3 and the plug 8.

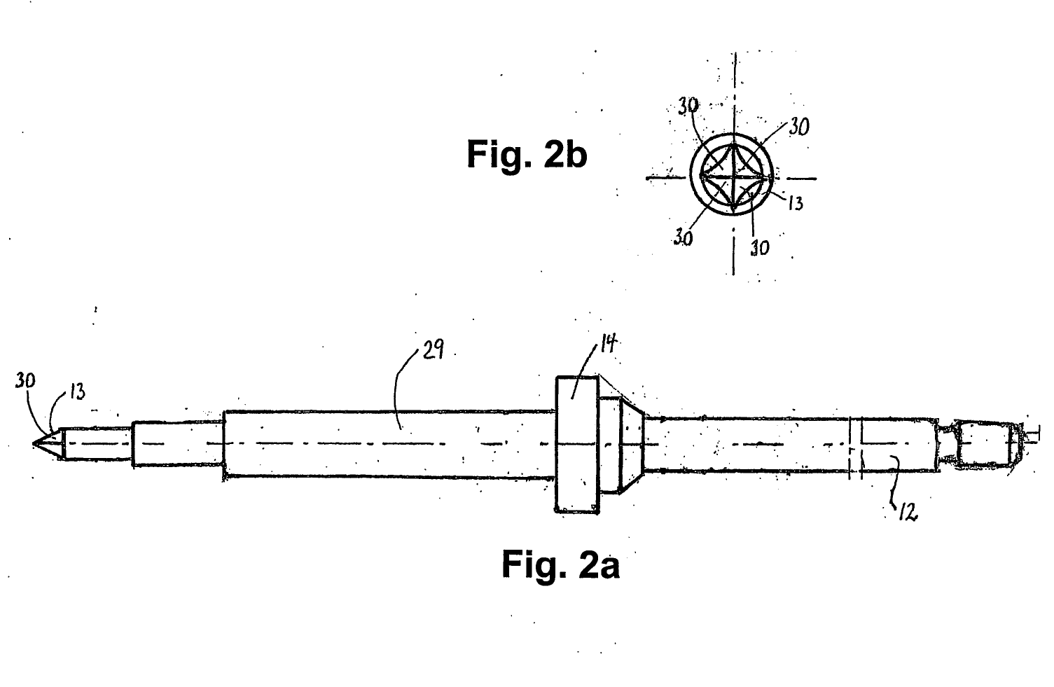

In FIG. 2 the rod 29 is shown in greater detail. The proximal end of the rod 29 is provided with a tip 13 having at least one recess 30. In FIG. 2b the tip is seen from the proximal end of the rod, showing 4 recesses 30. The rod 29 is furthermore provided with a shoulder 14 capable of functioning as a stopper means controlling the advancing movement of the rod 29, when the rod 29 is activated.

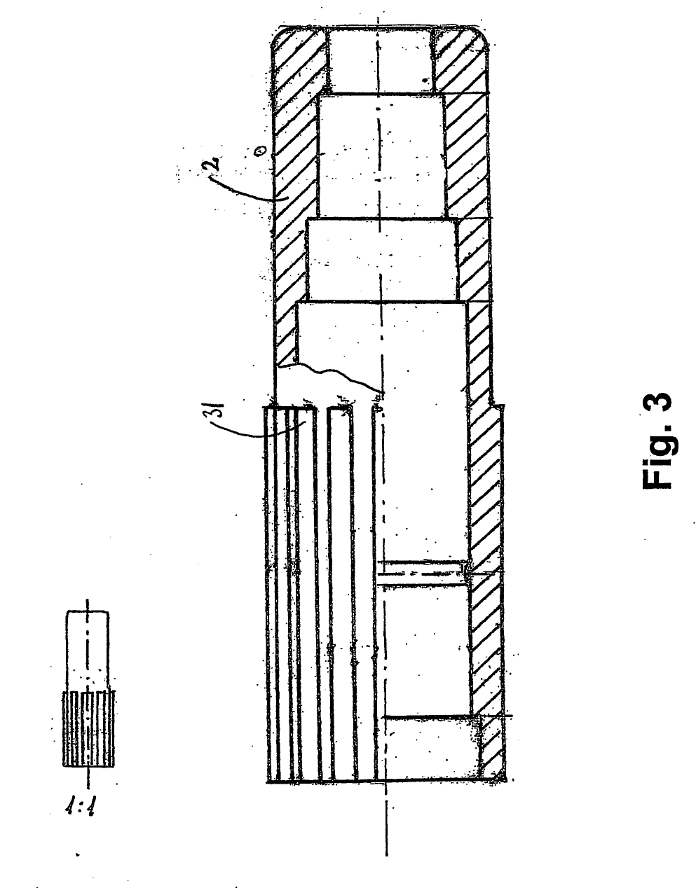

In FIG. 3, the housing 2 is shown in greater detail. The housing 2 is preferably provided with ribs 31 for a better grip of the device 1.

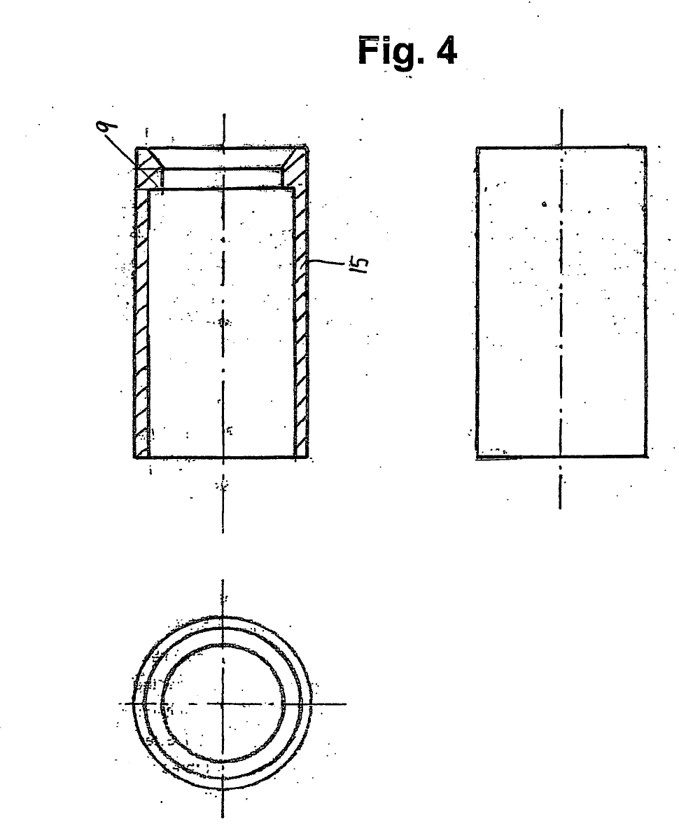

In FIG. 4 the internal bushing 15 is shown in greater detail. Projecting means 9 is arranged to engage the marker means when the rod 29 is activated.

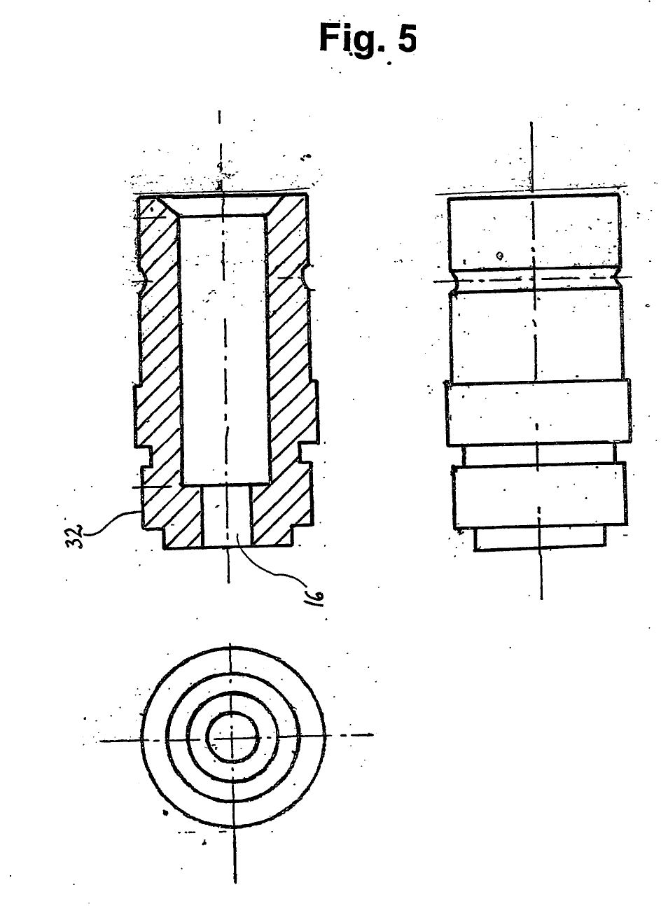

In FIG. 5 the bushing 11 is shown in greater detail. The bushing 11 is arranged in the proximal end of the housing 2 in the device 1 projecting from the proximal end. The rod 29 is capable of sliding through opening 16 of bushing 11. Furthermore, bushing 11 is provided with a projection part 32 capable of engaging the chamber house 3 when the chamber house 3 and the rod house are connected in a snap fit lock.

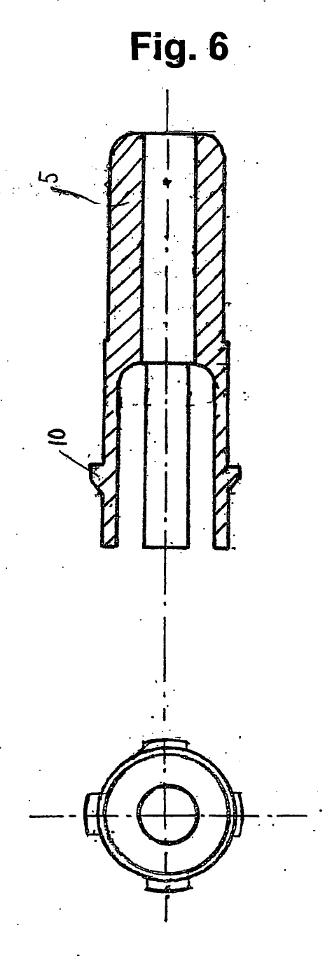

In FIG. 6 the marker means 5 is shown in greater detail. The marker means is provided with a central bore for receiving the rod 29, when assembled. The marker means is also provided with a projecting part 10 capable of engaging the projecting means 9 on the internal bushing 15.

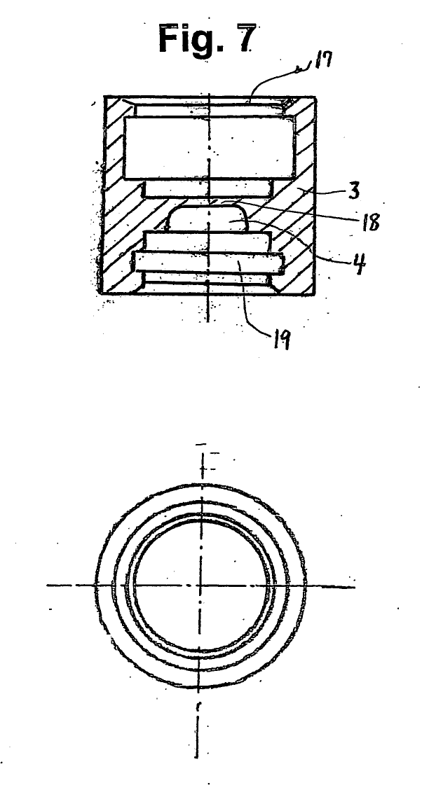

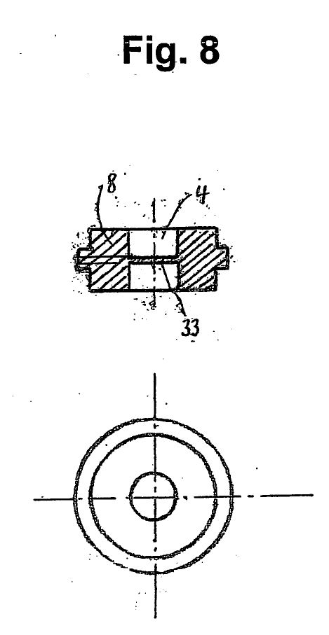

In FIG. 7, the chamber house 3 is shown in greater detail. The chamber house 3 comprises recess 17 capable of housing the proximal part of bushing 11 when the chamber house 3 is connected to the rod house. Furthermore, the chamber house 3 comprises a first sealing 18 as well as chamber 4. Recess 19 is arranged to receive a plug for providing the chamber house 3 with a second sealing. Plug 8 is shown in FIG. 8 having second sealing 33, whereby the plug 8 when inserted into the chamber house 3 encloses the chamber 4.

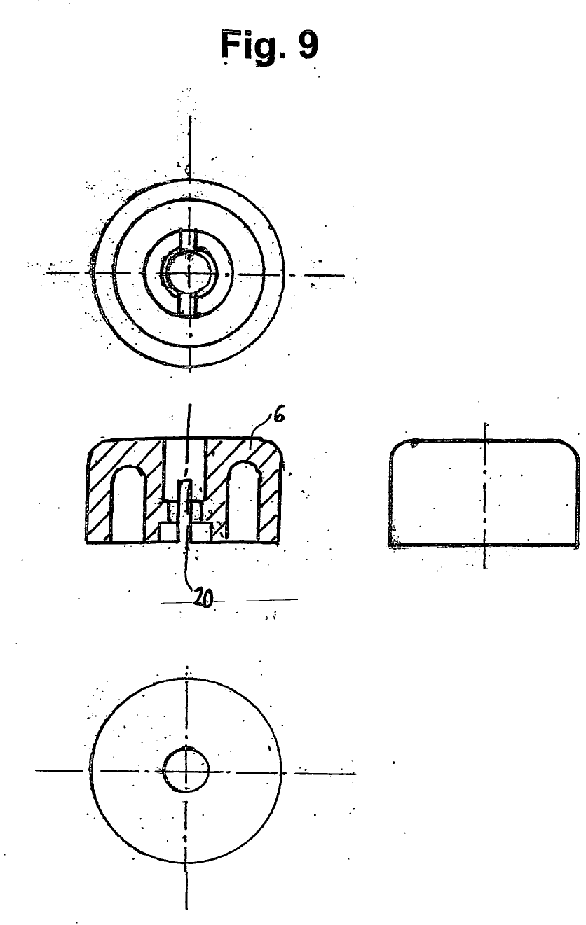

The cap 6 is shown in FIG. 9, wherein the recess 20 is capable of receiving the distal end of the rod 12.

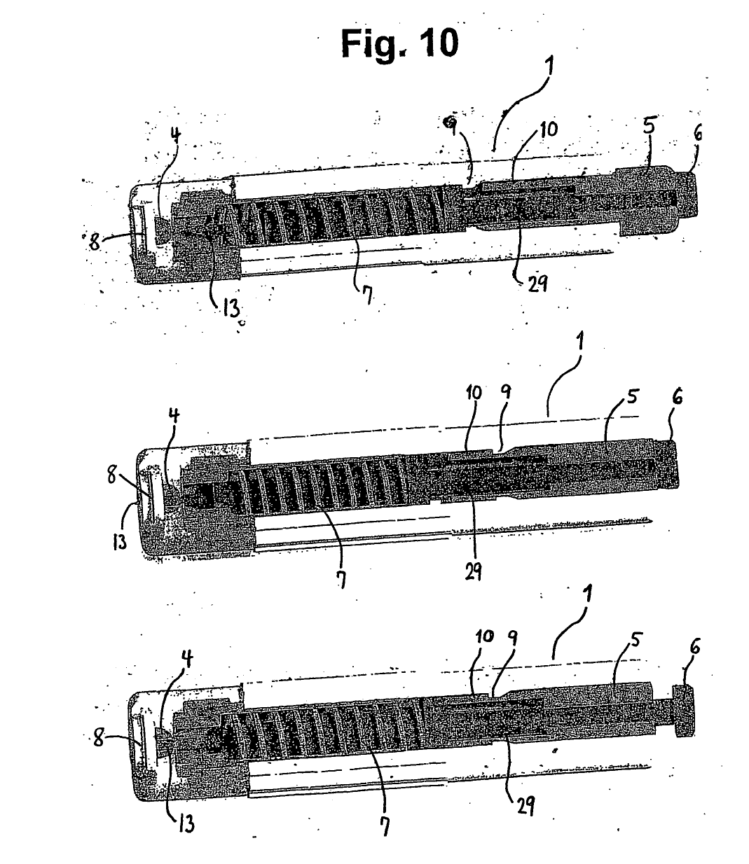

In FIG. 10 the travelling of the rod before, during and after activation is shown. In FIG. 10a the rod 29 is positioned in its inactivated position. The marker means 5 is located around the distal end of the rod and projects from the distal end of the rod house. The cap 6 is arranged on the distal end of the rod 29. The spring 7 is relaxed in the rod house. The tip 13 of the rod is position in the bushing 11, and the chamber 4 is filled with medicament or diagnostic agent; During activation of the rod 29, the rod 29 advances towards the proximal end of the device 1, penetrates the sealings of the chamber 4 and projects from the proximal end of the chamber house transferring medicament or diagnostic agent in its tip recesses. The marker means 5 is pressed into the housing 2 whereby projecting part 10 engages projecting means 9 of the housing. After activation the spring 7 forces the rod 29 backwards towards the distal end of the device 1 thereby the tip of the rod 29 is retracted into the chamber house 3. The marker means 5 is maintained engaged in the rod house, thereby signalling that the rod 29 has been activated and should not be used anymore.

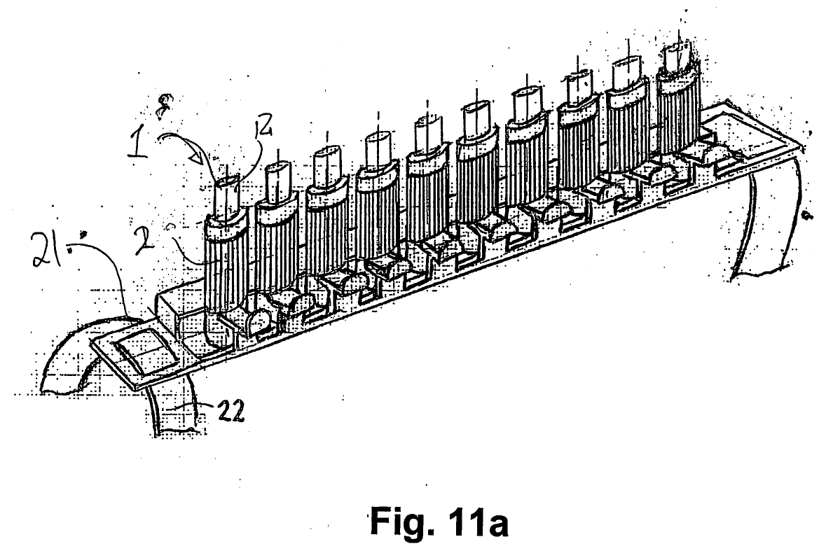

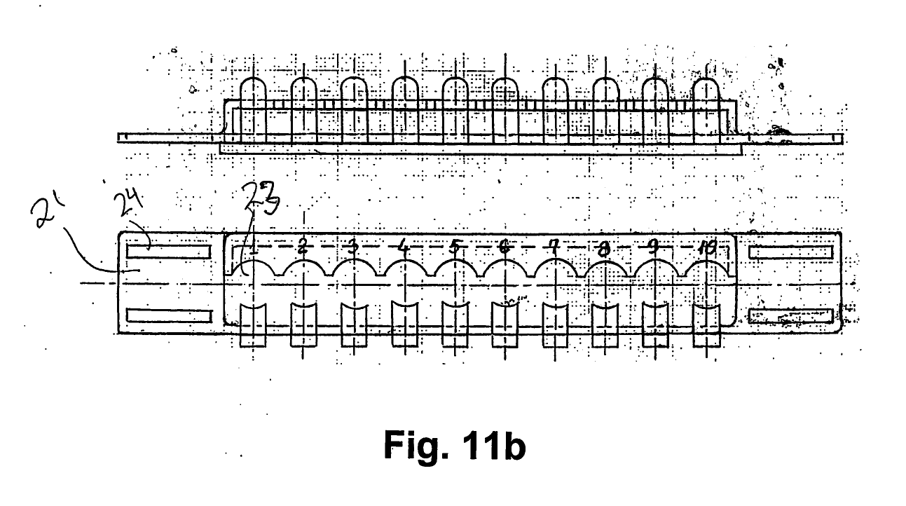

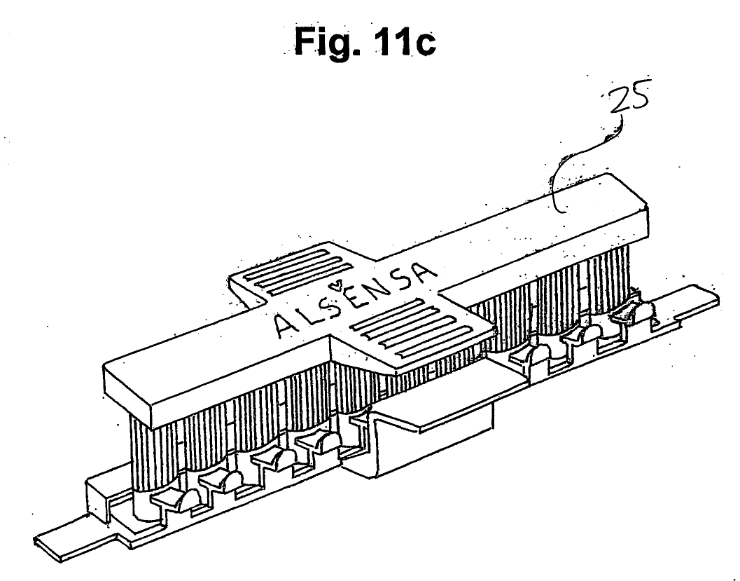

In FIG. 11a a multidevice according to the invention is shown. The multidevice comprises 10 devices 1 being arranged in a connector 21. Each device 1 is position perpendicular to the connector 21. The distal end 12 of the rod of the device is projecting from the connector 21. The connector 21 may be fastened to the animal to be tested by means of a bracelet 22. In FIG. 11b the opening for receiving a device 1 in the connector 21 is shown. Furthermore, the connector 21 is provided with recess 24 for receiving a bracelet 22.

In FIG. 11c the multidevice of FIG. 11a is shown having a connector cap 25 arranged above each and every distal end of the rods, so that all rods may be activated by activating the connector cap 25.

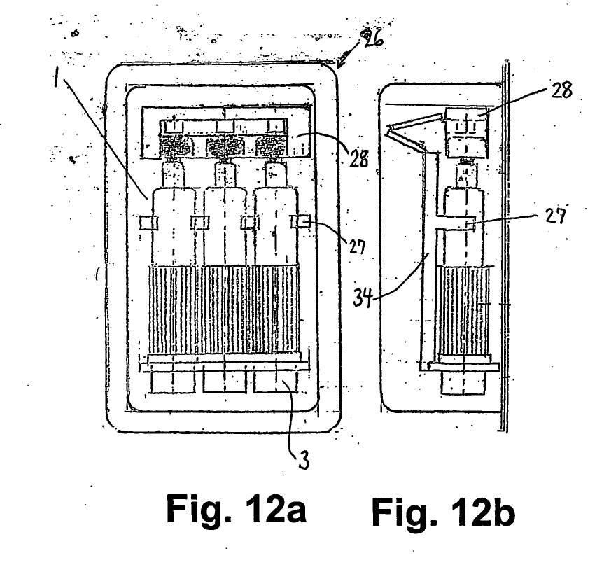

In FIG. 12a a package of a multidevice comprising 3 devices 1 is shown. The 3 devices 1 are connected through a holder 27 and a connector cap 28, wherein the holder 27 and the connector cap 28 are connected by means of stem 34 which is shown in FIG. 12b. The 3 chamber houses 3 are also connected, whereby the 3 chamber houses 3 may be provided as one unit.

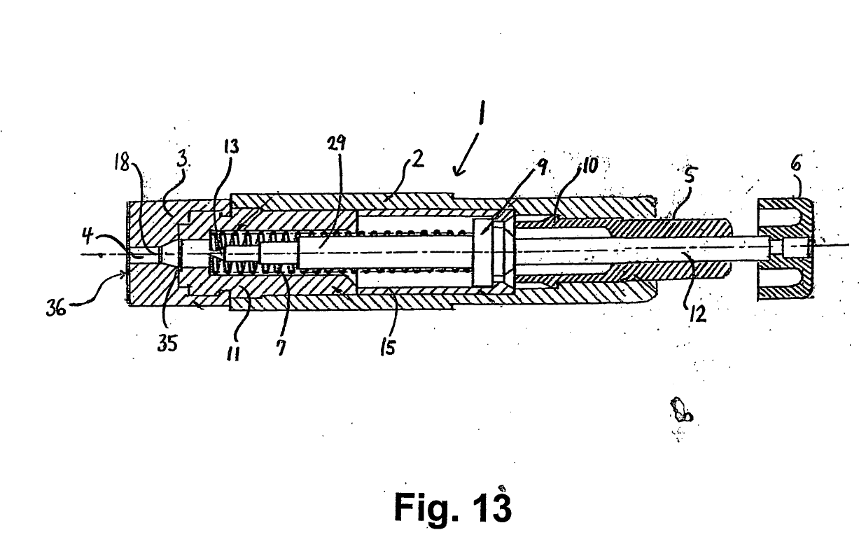

In FIG. 13 a device 1 according to the invention is shown. The device 1 is assembled of a rod house comprising a housing 2 and a rod 29, and a chamber house 3 comprising chamber 4. In the shown embodiment the rod house consists of housing 2, an internal bushing 15, and ambushing 11. The bushing 11 is closed by sealing 35 that has been moulded with the rod house. The rod house further encloses rod 29 arranged axially in the housing 2, spring 7 arranged concentrically around rod 29, as well as marker means 5 arranged concentrically around the distal end 12 of the rod 29. Furthermore, the distal end of the rod 29 is provided with a cap 6 facilitating the activation of the rod 29. In the proximal end of the device 1 the chamber house 3 is connected to the rod house through a snap fit lock to the bushing 11. The chamber house 3 has chamber 4 that is closed by cover 36, defining a chamber 4 between one wall of the chamber house 3 and the cover 36.

1. A device for delivering a medicament or a diagnostic agent to the skin or mucosa of an animal, said device comprising
at least one rod house and at least one separate chamber house, wherein said chamber house is capable of being connected to the rod house,
wherein the rod house comprises at least one housing, said at least one housing having a distal end and a proximal end, and at least one rod, said rod having a distal end and at least one proximal end, and said rod being slidably arranged in the housing, said rod being capable of being activated by being pushed towards the proximal end of the housing, and
wherein the chamber house comprises at least one chamber, a first wall of said chamber being a first sealing and a second wall of said chamber being a second sealing, said first sealing and said second sealing being arranged so that an axis through said chamber may intersect both sealings, and said chamber comprising said medicament or said diagnostic agent.

2. The device according to claim 1, wherein the rod is a longitudinal rod arranged axially in the rod house.

3. The device according to claim.1 or 2, wherein the rod house is sealed by a removable rod house sealing in the proximal end.

4. The device according to any of the preceding claims, wherein the distal end of the rod is projecting out of the distal end of the rod house.

5. The device according to any of the preceding claims, wherein the proximal end of the rod is a needle having a tapering end.

6. The device according to any of the preceding claims, wherein the needle comprises at least one recess in the tapering portion of the needle, such as at least two recesses in the tapering portion of the needle, such as at least four recesses in the tapering portion of the needle,

7. The device according to any of the preceding claims, wherein the chamber house is connected to the rod house so that the proximal end of the rod penetrates the first sealing wand the second sealing when slided proximally.

8. The device according to any of the preceding claims, wherein at least one marking means is arranged in the rod house to mark that the rod has been activated.

9. The device according to any of the preceding claims, wherein the at least one marking means is a marker projecting from the distal end of the housing and said marking means is activated when the rod is activated.

10. The device according to any of the preceding claims, wherein the at least one marking means is a marker being arranged concentrically around at least a part of the distal end of the rod.

11. The device according to any of the preceding claims, wherein the marking means is coloured in a colour different from the colour of the distal end of the rod.

12. The device according to any of the preceding claims, wherein the housing comprises means for engaging the marker means when the marker means is activated.

13. The device according to any of the preceding claims, wherein means for retracting the rod after activation is arranged in the housing.

14. The device according to any of the preceding claims, wherein the means for retracting the rod is a spring.

15. The device according to any of the preceding claims, wherein the housing comprises stopper means for stopping advance of the rod at a predetermined position during activation.

16. The device according to claim 15, wherein the rod has a shoulder and the stopper means is a shoulder in the housing dimensioned to engage the shoulder on the rod.

17. The device according to any of the preceding claims, wherein the chamber house has means for being attached to the rod house.

18. The device according to any of the preceding claims, wherein the second sealing is releasably attached to the chamber house.

19. The device according to any of the preceding claims, wherein the chamber house is made from a plastic material.

20. The device according to any of the preceding claims, wherein the chamber house is made from a resilient material.

21. The device according to any of the preceding claims, wherein the chamber house is attachable to the rod house by means of a thread or snap fit lock.

22. The device according to any of the preceding claims, wherein the rod has at least two proximal ends, such as at least three proximal ends.

23. The device according to any of the preceding claims, wherein the rod house comprises at least two rods, such as at least three rods.

24. The device according to claim 23, wherein the rods are connected at their distal ends to a common activation means.

25. The device according to any of the preceding claims, wherein the device comprises at least two chamber houses, such as at least three chamber houses.

26. The device according to claim 25, wherein at least two chamber houses are connected.

27. The device according to any of the preceding claims, wherein the device is provided with a labelling means, so that the skin or the mucosa of said animal is labelled when the medicament or diagnostic agent is delivered to the animal.

28. The device according to any of the preceding claims, wherein the labelling means is arranged on the chamber house.

29. The device according to any of the preceding claims, wherein the at least one chamber is filed at least partly with medicament or diagnostic agent.

30. A method for delivering a sufficient amount of medicament or diagnostic agent to an animal in need thereof, comprising
arranging a device as defined in any of the claims 1-29, wherein the chamber house comprising the medicament or diagnostic agent is connected to the rod house, adjacent the skin or mucosa of said animal,
activating the rod of the device, thereby delivering said medicament or diagnostic agent to said animal.

31. The method according to claim 30, wherein the chamber house comprises an allergen or combination of allergens.

32. A chamber house for being connected to a rod house as defined in any of claims 1-29, wherein the chamber house comprises a chamber, a first wall of said chamber being a first sealing and a second wall of said chamber being a second sealing, said first sealing and said second sealing being arranged so that an axis through said chamber may intersect both sealings, and said chamber comprising a medicament or a diagnostic agent.

33. The chamber house according to claim 32, wherein the chamber is at least partly filled with an allergen or a combination of allergens.

34. A rod house comprising at least one housing, said at least one housing having a distal end and a proximal end, and at least one rod, said rod having a distal end and at least one proximal end, and said rod being slidably arranged in the housing, said rod being capable of being activated by being pushed towards the proximal end of the housing, and said rod house being capable of being connected to a chamber house as defined in any of the claims 1-29.

35. A device for delivering a medicament or a diagnostic agent to the skin or mucosa of an animal, said device comprising
at least one rod house and at least one chamber house,
wherein the rod house comprises at least one housing, said at least one housing having a distal end and a proximal end, and at least one rod said rod having a distal end and at least one proximal end, and said rod being slidably arranged in the housing, said rod being capable of being activated by being pushed towards the proximal end of the housing,
wherein the chamber house comprises at least one chamber, a first wall of said chamber being a first sealing and a second wall of said chamber being a second sealing, said first sealing and said second sealing being arranged so that an axis through said chamber may intersect both sealings, and said chamber comprising said medicament or said diagnostic agent, and
wherein the chamber house is provided with a labelling means for transferring a label to the animal being tested.

36. The device according to claim 35, wherein the device has one or more of the features of claims 1-29.
